SEQUENCE LISTING (16 sequences)

SEQ ID NO: 1 - DNA, 21 bp, synthetic construct
  source 1..21: mol_type = other DNA
acgggatagtgctcgaaatca

SEQ ID NO: 2 - DNA, 20 bp, synthetic construct
  source 1..20: mol_type = other DNA
gtctgcttggaatgcgagtg

SEQ ID NO: 3 - DNA, 22 bp, synthetic construct
  source 1..22: mol_type = other DNA
tggaggttcattaaatggcacg

SEQ ID NO: 4 - DNA, 23 bp, synthetic construct
  source 1..23: mol_type = other DNA
tcgtgatttagttacacaggcca

SEQ ID NO: 5 - DNA, 20 bp, synthetic construct
  source 1..20: mol_type = other DNA
cggtagctgaggaacccttc

SEQ ID NO: 6 - DNA, 20 bp, synthetic construct
  source 1..20: mol_type = other DNA
cctctcaggagccctggtaa

SEQ ID NO: 7 - DNA, 22 bp, synthetic construct
  source 1..22: mol_type = other DNA
tggatcagcaagcaggagtacg

SEQ ID NO: 8 - DNA, 22 bp, synthetic construct
  source 1..22: mol_type = other DNA
aggagggcaaagtggtaaacgc

SEQ ID NO: 9 - DNA, 20 bp, synthetic construct
  source 1..20: mol_type = other DNA
caggatgccaagccatgagg

SEQ ID NO: 10 - DNA, 20 bp, synthetic construct
  source 1..20: mol_type = other DNA
gcatcttctccagcaagtcg

SEQ ID NO: 11 - DNA, 21 bp, synthetic construct
  source 1..21: mol_type = other DNA
ctcctttaggtgcagcagtga

SEQ ID NO: 12 - DNA, 20 bp, synthetic construct
  source 1..20: mol_type = other DNA
gaggccaaagtcggatctgt

SEQ ID NO: 13 - DNA, 20 bp, synthetic construct
  source 1..20: mol_type = other DNA
tcctttgagccgtttggagg

SEQ ID NO: 14 - DNA, 20 bp, synthetic construct
  source 1..20: mol_type = other DNA
tacatactgccgcaggtcac

SEQ ID NO: 15 - DNA, 19 bp, synthetic construct
  source 1..19: mol_type = other DNA
ggagtcaacggatttggtc

SEQ ID NO: 16 - DNA, 22 bp, synthetic construct
  source 1..22: mol_type = other DNA
ggaatcattggaacatgtaaac

(19) United States
(12) Patent Application Publication
(10) Pub. No.: US 2025/0281561 A1
(43) Pub. Date: Sep. 11, 2025

(54) APPLICATION OF LYCIUM BARBARUM LEAF EXTRACT IN PREPARING DRUG FOR TREATING DIABETIC RETINOPATHY

(71) Applicant: NANJING UNIVERSITY OF CHINESE MEDICINE, Nanjing (CN)

(72) Inventors: Sheng GUO, Nanjing (CN); Jinao DUAN, Nanjing (CN); Qing LIU, Nanjing (CN); Hongjie KANG, Nanjing (CN); Shulan SU, Nanjing (CN)

(73) Assignee: NANJING UNIVERSITY OF CHINESE MEDICINE, Nanjing (CN)

(21) Appl. No.: 19/215,397

(22) Filed: May 22, 2025

(30) Foreign Application Priority Data
Apr. 17, 2025 (CN) 2025104835249

(51) Int. Cl. A61K 36/815; A61P 3/10; A61P 27/02

(52) U.S. Cl. CPC A61K 36/815; A61P 3/10; A61P 27/02; A61K 2236/331; A61K 2236/333; A61K 2236/39; A61K 2236/51

(57) ABSTRACT

Disclosed is an application of a Lycium barbarum leaf extract in preparing a drug for treating diabetic retinopathy. According to the invention, a refined Lycium barbarum leaf extract is prepared by specific process steps, and active ingredients of Lycium barbarum leaf can protect ARPE-19 cells from high glucose-induced damage, have a certain improvement effect on zebrafish with diabetic retinopathy, and significantly improve eye symptoms in a diabetic retinopathy mouse model. The Lycium barbarum leaf extract provided by the invention has the potential to be a drug for treating diabetic retinopathy.

### REFERENCE TO AN ELECTRONIC SEQUENCE LISTING

The contents of the electronic sequence listing (sequencelisting: 250511.xml; Size: 15,032 bytes; and Date of Creation: Friday, May 9, 2025) is herein incorporated by reference in its entirety.

### CROSS-REFERENCE TO RELATED APPLICATIONS

This application claims priority to Chinese Patent Application Ser. No. CN2025104835249 filed on 17 Apr. 2025.

### TECHNICAL FIELD

The present invention relates to a traditional Chinese medicine, and particularly to an application of a Lycium barbarum leaf extract in improving diabetic retinopathy, and belongs to the technical field of medicine.

### BACKGROUND

Diabetes is a common disease, and there are many complications in the middle and advanced stage of diabetes, wherein diabetic retinopathy (DR), as one of the complications of diabetes, is one of the main causes of blindness. Effective prevention and treatment of the occurrence and development of DR has become an urgent need of society. The pathogenesis of DR is complex. At present, it is generally believed that the pathogenesis of DR comprises hyperglycemia-induced microangiopathy, neuronal lesion and moderate and low inflammatory reaction. The clinical treatment of DR mainly comprises control of blood glucose and blood lipid, laser photocoagulation, and antibody drug injection into a vitreous body. In recent years, traditional Chinese medicine has shown obvious characteristics and advantages in delaying the progress of DR, reducing the incidence of DR and improving the quality of life of patients with DR because of its mild and lasting effects, multiple components and multiple target points. Therefore, under the guidance of theory of traditional Chinese medicine, the research and development of new drug for preventing and treating the DR has become a research hotspot at present.

Lycium barbarum leaves (named “Tianjingcao” in “Compendium of Materia Medica”) are dried tender leaves of Lycium barbarum L. or L. chinense Mill., which have the functions of tonifying deficiency and boosting essence, clearing away heat, quenching thirst, and expelling wind and improving eyesight, and have many pharmacological effects, such as immunomodulation, anti-tumor, anti-oxidation, and anti-aging effects, and decrease of blood glucose and blood lipid.

There is no report on the improvement of DR by different active ingredients of Lycium barbarum leaf and its potential mechanism.

### SUMMARY

Objective of invention: the present invention aims to screen and obtain active ingredients of Lycium barbarum leaf with the best curative effect by studying improvement effects of different active ingredients of Lycium barbarum leaf on DR, and to provide theoretical basis for the rational use of Lycium barbarum leaf to treat DR in clinic.

In the present invention, different active ingredients of Lycium barbarum leaf will be used to treat ARPE-19 cells, A/B-type zebrafish and C57BL/6J mice, and protective effects of different active ingredients of Lycium barbarum leaf on ARPE-19 cells with high glucose-induced damage are investigated by methods such as a CCK8, a qPCR, an oxidation kit (MDA, SOD, and GSH), and ROS; and protective effects of different active ingredients of Lycium barbarum leaf on a DR zebrafish model are investigated by the detection of glucose level in DR zebrafish, a change of retinal vascular diameter in zebrafish, a qPCR, and an oxidation kit (MDA, SOD, and GSH), so as to screen and obtain the best active ingredients of Lycium barbarum leaf to improve DR.

In the present invention, various active ingredients of Lycium barbarum leaf are obtained by the following method: 1 kg of dried Lycium barbarum leaves are weighed, added with distilled water 10 times the mass, heated and refluxed for extraction twice, for 1 hour each time, concentrated under a reduced pressure, and freeze-dried to obtain a Lycium barbarum leaf water extracting solution;
2 kg of dried Lycium barbarum leaves are weighed, added with 80% ethanol 10 times the mass, and heated and refluxed for extraction twice, for 1 hour each time, to obtain a Lycium barbarum leaf ethanol extracting solution and an ethanol extracting herb residue; the ethanol extracting solution is uniformly divided into 2 parts, wherein one part is concentrated under a reduced pressure, freeze-dried, and then used as a Lycium barbarum leaf ethanol extract, and the other part is concentrated to a suitable volume under a reduced pressure, then fully absorbed with D101 macroporous resin for 24 hours, and eluted with distilled water, 10% ethanol and 30% ethanol for 2-3 column volumes respectively, an eluate is discarded, the rest is eluted with 70% ethanol which serves as an eluent for 2-3 column volumes, then an eluate is collected, concentrated under a reduced pressure, and freeze-dried to obtain a refined Lycium barbarum leaf extract; and
the ethanol extracting herb residue is air-dried, then added with distilled water 10 times the mass, heated and refluxed for extraction twice, for 1 hour each time, concentrated under a reduced pressure, then added with anhydrous ethanol until a concentration of ethanol is 80%, and allowed to stand at 4° C. overnight, a supernatant is discarded, the rest is dried to obtain a crude polysaccharide, and the crude polysaccharide is deproteinized by a Sevage (chloroform: n-butanol=4:1) method to obtain a refined polysaccharide.

Studies show that 98% of alkaloid in the Lycium barbarum leaves is betaine, so that the betaine is used as the alkaloid in the Lycium barbarum leaves for further study.

In the present invention, the refined Lycium barbarum leaf extract and a pharmaceutically acceptable carrier are made into a drug in a form of a tablet, a capsule, a granule or a pill.

In the present invention, when the tablet is made, the refined Lycium barbarum leaf extract is added with carrier lactose or corn starch, added with a lubricant magnesium stearate when necessary, and mixed evenly, and then the mixture is pressed to make the tablet.

When the capsule is made, the refined Lycium barbarum leaf extract and the carrier lactose or corn starch are evenly mixed, granulated, and then encapsulated to make the capsule.

When the granule is made, the refined Lycium barbarum leaf extract and the diluent lactose or corn starch are evenly mixed, granulated and dried to make the granule.

Beneficial effects: the Lycium barbarum leaf extract provided by the present invention has the following advantages compared with the prior art:

In the present invention, improvement effects of different active ingredients of Lycium barbarum leaf on a DR in-vitro cell model and a zebrafish model are screened, and verified in an in-vivo animal model, and experimental results show that, except the Lycium barbarum leaf polysaccharide, the Lycium barbarum leaf water extract, the Lycium barbarum leaf ethanol extract, the refined Lycium barbarum leaf extract, and the Lycium barbarum alkaloid active ingredient all can improve a survival rate of ARPE-19 cells with high glucose-induced damage, down-regulate expression levels of ERK, p38 and JNK, improve oxidative stress, and significantly reduce the generation of ROS. Compared with other groups, the refined Lycium barbarum leaf extract has a more prominent effect under the same crude drug dosage, which indicates that the refined Lycium barbarum leaf extract is the main active ingredient.

In a DR zebrafish model, except the Lycium barbarum leaf polysaccharide, the Lycium barbarum leaf water extract, the Lycium barbarum leaf ethanol extract, the refined Lycium barbarum leaf extract, and the Lycium barbarum alkaloid active ingredient all can reduce a glucose content in vivo, down-regulate expression levels of ERK, p38 and JNK, improve oxidative stress, and significantly reduce a retinal vascular diameter of zebrafish.

Compared with other groups, the refined Lycium barbarum leaf extract has a more prominent effect under the same crude drug dosage, which indicates that the refined Lycium barbarum leaf extract is the main active ingredient.

Based on the in-vitro cell model and the zebrafish model, compared with the Lycium barbarum leaf water extract and the Lycium barbarum leaf ethanol extract, the results show that the refined Lycium barbarum leaf extract has a more prominent effect under the same crude drug dosage, which indicates that the ingredients of the refined Lycium barbarum leaf extract are the best active ingredients to improve the DR, so that an improvement effect of the refined Lycium barbarum leaf extract on the DR is further verified in a DR mouse model, and by detecting a body weight change, a blood glucose level, a retinal diameter, and a retinal vascular density of mice, it is proved that the refined Lycium barbarum leaf extract can significantly increase the retinal vascular diameter of mice and reduce the retinal vascular density of mice, so that the refined Lycium barbarum leaf extract screened and obtained by the present invention may be used as a drug for treating and improving the DR. The present invention can realize efficient and comprehensive utilization of Lycium barbarum leaf resources.

### BRIEF DESCRIPTION OF THE DRAWINGS

FIG. 1 shows effects of different active ingredients of Lycium barbarum leaf on a survival rate (CCK8) and an oxidative stress level (GSH, SOD, and MDA) of ARPE-19 cells with high glucose-induced damage.

FIG. 2 shows effects of different active ingredients of Lycium barbarum leaf on a gene expression level (p38, ERK, and JNK) of the ARPE-19 cells with high glucose-induced damage.

FIG. 3A shows effects of different active ingredients of Lycium barbarum leaf on a ROS content in the ARPE-19 cells with high glucose-induced damage: a representative picture of ROS contents after administration of various active ingredients;

FIG. 3B shows effects of different active ingredients of Lycium barbarum leaf on a ROS content in the ARPE-19 cells with high glucose-induced damage: shows statistical results of ROS content levels.

FIG. 4 shows effects of different active ingredients of Lycium barbarum leaf on a glucose content (GLU) and an oxidative stress level (GSH, SOD, and MDA) in DR zebrafish.

FIG. 5 shows effects of different active ingredients of Lycium barbarum leaf on a gene expression level (p38, ERK, and JNK) in the DR zebrafish.

FIG. 6A shows effects of different active ingredients of Lycium barbarum leaf on a retinal vascular diameter in the DR zebrafish: a representative picture of retinal vessels of the zebrafish after administration of various active ingredients.

FIG. 6B shows effects of different active ingredients of Lycium barbarum leaf on a retinal vascular diameter in the DR zebrafish: statistical results of retinal vascular diameters after administration of various active ingredients.

FIG. 7A shows effects of different active ingredients of Lycium barbarum leaf on a body weight and a blood glucose level of DR mice: body weight changes of the mice after administration of various active ingredients.

FIG. 7B shows effects of different active ingredients of Lycium barbarum leaf on a body weight and a blood glucose level of DR mice: blood glucose level changes of the mice after administration of various active ingredients.

FIG. 8A shows effects of different active ingredients of Lycium barbarum leaf on a retinal vascular density of the DR mice: a representative picture of retinal vascular densities of the mice after administration of various active ingredients.

FIG. 8B shows effects of different active ingredients of Lycium barbarum leaf on a retinal vascular density of the DR mice: statistical results of retinal vascular densities of the mice after administration of various active ingredients.

FIG. 9A shows effects of different active ingredients of Lycium barbarum leaf on a retinal vascular diameter in the DR mice: a representative picture of retinal vascular diameters of the mice after administration of various active ingredients.

FIG. 9B shows effects of different active ingredients of Lycium barbarum leaf on a retinal vascular diameter in the DR mice: statistical results of retinal vascular diameters of the mice after administration of various active ingredients.

### DETAILED DESCRIPTION

The present invention can be better understood according to the following embodiments. However, it is easy for those skilled in the art to understand that specific material ratios, process conditions and results described in the embodiments are only used to illustrate the present invention, and shall not and will not limit the present invention described in detail in the claims.

The reagents used in the embodiments of the present invention comprise:

| Name of reagent | Manufacturer |
|---|---|
| Fetal bovine serum (FBS) | Gibco Company in America |
| Penicillin | BI Company in Israel |
| Streptomycin | BI Company in Israel |
| DMEM/F12 culture medium | Gibco Company in America |
| CCK8 detection kit | Shanghai Beyotime Biotech Inc |
| H2FDCA | Glpbio Company in America |

The primers used in the embodiments of the present invention comprise:

| Name of gene | Sequence of primer |
|---|---|
| p38 (zebrafish) | F: ACGGGATAGTGCTCGAAATCA |
| | (SEQ ID NO: 01) |
| | R: GTCTGCTTGGAATGCGAGTG |
| | (SEQ ID NO: 02) |
| JNK (zebrafish) | F: TGGAGGTTCATTAAATGGCACG |
| | (SEQ ID NO: 03) |
| | R: TCGTGATTTAGTTACACAGGCCA |
| | (SEQ ID NO: 04) |
| ERK (zebrafish) | F: CGGTAGCTGAGGAACCCTTC |
| | (SEQ ID NO: 05) |
| | R: CCTCTCAGGAGCCCTGGTAA |
| | (SEQ ID NO: 06) |
| ß-actin | F: TGGATCAGCAAGCAGGAGTACG |
| (zebrafish) | (SEQ ID NO: 07) |
| | R: AGGAGGGCAAAGTGGTAAACGC |
| | (SEQ ID NO: 08) |
| p38 (ARPE-19) | F: CAGGATGCCAAGCCATGAGG |
| | (SEQ ID NO: 09) |
| | R: GCATCTTCTCCAGCAAGTCG |
| | (SEQ ID NO: 10) |
| JNK (ARPE-19) | F: CTCCTTTAGGTGCAGCAGTGA |
| | (SEQ ID NO: 11) |
| | R: GAGGCCAAAGTCGGATCTGT |
| | (SEQ ID NO: 12) |
| ERK (ARPE-19) | F: TCCTTTGAGCCGTTTGGAGG |
| | (SEQ ID NO: 13) |
| | R: TACATACTGCCGCAGGTCAC |
| | (SEQ ID NO: 14) |
| GAPDH (ARPE-19) | F: GGAGTCAACGGATTTGGTC |
| | (SEQ ID NO: 15) |
| | R: GGAATCATTGGAACATGTAAAC |
| | (SEQ ID NO: 16) |

The animals and cells used in the embodiments of the present invention comprise:

ARPE-19 cells came from an ATCC cell bank, cultured with a DMEM/F12 complete culture medium (containing 1% double-antibody) containing 10% FBS in a cell incubator (37° C., 5% CO₂), and passaged once every 2-3 days;

wild A/B-type zebrafish and fli1: EGFP zebrafish, 6 months old, purchased from Nanjing YSY Biotech Co., Ltd., and cultivated in 28.5° C. circulating filtration water system according to illumination time of 14 hours (light): 10 hours (dark), wherein zebrafish embryos with normal development 2 hours after fertilization are taken for follow-up experiment; and

C57BL/6J mice, 8 weeks old, purchased from Shanghai Slake Experimental Animal Co., Ltd., and fed in cages by groups, wherein the blank group is fed with ordinary feed and the other groups are fed with high-glucose and high-fat feed under the conditions of free eating and drinking, normal illumination, and temperature controlled at 20-25° C. by operations and research processes that are all carried out in accordance with the “Regulations for the Administration of Affairs Concerning Experimental Animals”.

### Embodiment 1: Preparation of Active Ingredients of Lycium barbarum Leaf

1 kg of dried Lycium barbarum leaves were weighed, added with distilled water 10 times the mass, heated and refluxed for extraction twice, for 1 hour each time, concentrated under a reduced pressure, and freeze-dried to obtain a Lycium barbarum leaf water extract.

2 kg of dried Lycium barbarum leaves were weighed, added with 80% ethanol 10 times the mass, and heated and refluxed for extraction twice, for 1 hour each time, to obtain a Lycium barbarum leaf ethanol extracting solution and an ethanol extracting herb residue; and the ethanol extracting solution was uniformly divided into 2 parts. One part was concentrated under a reduced pressure, freeze-dried, and then used as a Lycium barbarum leaf ethanol extract.

The other part was concentrated to a suitable volume under a reduced pressure, then fully absorbed with D101 macroporous resin for 24 hours, and eluted with distilled water, 10% ethanol and 30% ethanol for 2-3 column volumes respectively, an eluate was discarded, the rest was eluted with 70% ethanol which serves as an eluent for 2-3 column volumes, then an eluate was collected, concentrated under a reduced pressure, and freeze-dried to obtain a refined Lycium barbarum leaf extract.

The Lycium barbarum leaf herb residue after ethanol extraction was air-dried, then added with distilled water 10 times the mass, heated and refluxed for extraction twice, for 1 hour each time, concentrated under a reduced pressure, then added with anhydrous ethanol until a concentration of ethanol was 80%, and allowed to stand at 4° C. overnight, a supernatant was discarded, the rest was dried to obtain a crude polysaccharide, and the crude polysaccharide was deproteinized by a Sevage (chloroform: n-butanol=4:1) method to obtain a Lycium barbarum polysaccharide. Studies show that 98% of alkaloid in the Lycium barbarum leaves is betaine, so that the betaine is used as the alkaloid in the Lycium barbarum leaves for further study.

### Embodiment 2: Activity Screening Based on Cell Model

### 1. Experimental Method

1.1 ARPE-19 Cells with High-Glucose Induced Damage

Preparation method of active ingredient solution of Lycium barbarum leaf: 1 mg of freeze-dried powder of different active ingredients of Lycium barbarum leaf (Lycium barbarum leaf water extract, Lycium barbarum leaf ethanol extract, refined Lycium barbarum leaf extract, Lycium barbarum polysaccharide, and betaine) prepared in Embodiment 1 was weighed respectively, and dissolved in 1 mL of DMEM/F12 culture medium to prepare 1 mg/mL solution, and the solution was diluted to a required concentration with DMEM/F12 culture medium during administration, and filtered with a 22 μm filter for sterilization.

The ARPE-19 cells were cultured in DMEM/F12 culture medium containing 10% FBS and 1% penicillin/streptomycin at 37° C. under 5% CO₂. A model group was treated with 300 mM glucose, and the culture medium was added with a corresponding drug to culture the cells for 48 hours to complete the modeling.

1.2 Kit Detection of Protective Effects of Active Ingredients of Lycium barbarum Leaf on ARPE-19 Cells with High-Glucose Induced Damage

The cells were inoculated into a 96-well plate during culture, and divided into a control group (Con group), a model group (Mod group), a high-dose Lycium barbarum leaf water extract group (ST-H group), a low-dose Lycium barbarum leaf water extract group (ST-L group), a high-dose Lycium barbarum leaf ethanol extract group (CT-H group), a low-dose Lycium barbarum leaf ethanol extract group (CT-L group), a high-dose refined Lycium barbarum leaf extract group (FT-L group), a low-dose Lycium barbarum polysaccharide group (DT-L group), a high-dose betaine group (SWJ-H group), and a low-dose betaine group (SWJ-L group), wherein the high dose was 10 μg/mL and the low dose was 1 μg/mL. After the culture was completed, the culture medium was discarded, and 10% CCK8 solution was added to incubate the cells at 37° C. for 1 hour. An absorbance was measured at 450 nm, and a cell survival rate was calculated.

The cells were inoculated into a 6-well plate during cell culture, and divided into a Con group, a Mod group, a ST-H group, a ST-L group, a CT-H group, a CT-L group, a FT-H group, a FT-L group, a DT-H group, a DT-L group, a SWJ-H group, and a SWJ-L group, wherein the high dose was 10 μg/mL and the low dose was 1 μg/mL. After the culture was completed, adherent cells were collected, centrifuged at 4° C. and 800 g/min for 5 minutes, washed with PBS twice, centrifuged again, and added with 300 ml of PBS, ultrasonic treatment was turned on for 2 seconds and turned off for 8 seconds, and the cells were disrupted in ice water bath for 2 minutes, and centrifuged at 4° C. and 4000 rpm/min for 10 minutes. A GSH level, and SOD and MDA activities in the cells were detected according to a kit instruction.

1.3 qPCR detection of effects of active ingredients of Lycium barbarum leaf on gene expression level of ARPE-19 cells with high glucose-induced damage

### (1) Extraction of Total RNA

The cells were inoculated into a 6-well plate during cell culture, and divided into a Con group, a Mod group, a ST group, a CT group, a FT group, a DT group, and a SWJ group, wherein the administration dosage was 10 μg/mL. The cells were collected into Ep tubes and centrifuged at 1000 rpm for 5 minutes, and a supernatant was discarded. Each tube was added with 1 ml of Trizol reagent and blown violently for 15 seconds to mix fully and evenly, and the mixture was allowed to stand at room temperature for 2-3 minutes, and rotated at 4° C. for 1-2 hours. 200 μL of chloroform was added, and vortexed for about 15 seconds, until the liquid had no obvious stratification, and then the liquid was centrifuged at 12000 g for 15 minutes. 500 μL of supernatant was sucked and added into a ribozyme-free Ep tube, added with 500 μL of isopropanol, vortexed to mix fully and evenly, and then the mixture was put on ice, incubated for 5-10 minutes, and centrifuged at 12000 g for 10 minutes. A supernatant was discarded, and 1 mL of 75% ethanol (precooled at 4° C. in advance) prepared from DEPC water was added, flicked to mix evenly, and centrifuged at 7500 g for 5 minutes. A supernatant was discarded, the remaining ethanol was evaporated in a fuming hood, added with 25 μL of EPC water, and flicked to mix evenly, and the mixture was put on ice. A concentration was determined by an ultra-micro ultraviolet spectrophotometer, and then the mixture was stored in a refrigerator at −80° C. for later use.

(2) Synthesis of cDNA

The cDNA was synthesized according to the kit instruction, and the synthesis system was as shown in Table 4. A reverse transcription procedure was as follows: the system was incubated at 42° C. for 15 minutes, and inactivated at 95° C. for 3 seconds. The product was stored in a refrigerator at −20° C. for later use.

TABLE 4
Synthesis system of cDNA

| Reagent | Volume (μL) |
|---|---|
| RNA | 5 |
| Random Primer | 0.5 |
| Anchored Oligo (dT) 18 Primer | 0.5 |
| E-Mix | 1 |
| R-Mix | 1 |
| gDNA Remover (2×) | 10 |
| RNase-free Water | 2 |

### (3) PCR Amplification

A PCR amplification system was shown in Table 5. The reaction system was mixed fully and evenly, added to a 96-well PCR plate, centrifuged, and determined by a PCR machine. PCR reaction conditions referred to Table 6. β-actin was used as an internal reference. A CT value of a sample was determined, and then relative expressions of p38, JNK and ERK in the sample were calculated by a 2-ΔΔCT method.

TABLE 5
PCR amplification system

| Reagent | Volume (μL) |
|---|---|
| CDNA | 1 |
| SYBR Green (2×) | 25 |
| Forward primer | 1 |
| Reverse primer | 1 |
| RNase-free Water | 22 |

TABLE 6
PCR reaction conditions

| | | Number of | | | |
|---|---|---|---|---|---|
| Stage | Purpose | cycles | Temperature | Time | |
| 1 | Pre- | 1 | 50 | 2 | min |
| | denaturation | | 94 | 30 | s |
| 2 | | 45 | 94 | 5 | s |
| | Circular | | 60 | 34 | s |
| | reaction | | | | |
| 3 | Dissolution | 1 | 95 | 15 | s |
| | curve | | 60 | 1 | min |
| | | | 95 | 1 | s |

1.4 Effects of Active Ingredients of Lycium barbarum Leaf on ROS Content in ARPE-19 Cells with High Glucose-Induced Damage

The cells were inoculated into a 96-well plate during culture, and divided into a Con group, a Mod group, a ST-H group, a ST-L group, a CT-H group, a CT-L group, a FT-H group, a FT-L group, a DT-H group, a DT-L group, a SWJ-H group, and a SWJ-L group, wherein the high dose was 10 g/mL and the low dose was 1 μg/mL. After the culture was completed, the culture medium was discarded, and a culture medium containing 1/1000 H2FCDA was added to incubate the cells at 37° C. for 30 minutes. A photo was taken by a fluorescence microscope at an excitation wavelength of 488 nm, and a fluorescence intensity was analyzed by ImageJ.

### 1.5 Statistical Analysis

Data analysis and drawing were carried out by GraphPad Prism 9.4.0 software, all the results were expressed by mean±standard deviation (SD), data between groups were analyzed by one-way analysis of variance (One-way ANOVA), and the difference was statistically significant when P<0.05. (Compared with the model group, *P<0.05; ** P<0.01; *** P<0.001; and compared with the blank group, #P<0.05; ##P<0.01; ###P<0.001).

### 2. Experimental Results

2.1 Protective Effects of Active Ingredients of Lycium barbarum Leaf on ARPE-19 Cells with High-Glucose Induced Damage

In the present invention, the protective effects of different active ingredients of Lycium barbarum leaf on the ARPE-19 cells with high-glucose induced damage are investigated, and effects of different active ingredients of Lycium barbarum leaf on a survival rate and an oxidative stress level of the ARPE-19 cells with high-glucose induced damage are detected by a CCK8 detection kit and a MDA, SOD and GSH detection kit in combination with a microplate reader.

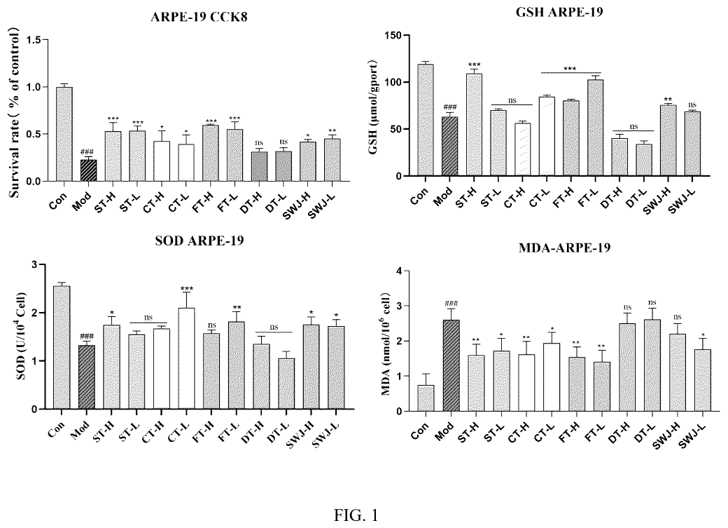

Detection results are as shown in FIG. 1, wherein the ordinates represent a cell survival rate, a GSH level, and MDA and SOD activities respectively. After high glucose damage, the survival rate, the GSH content, and the SOD activity of the ARPE-19 cells are all decreased significantly compared with the blank group, and the MDA activity is increased significantly. After administration, except the Lycium barbarum leaf polysaccharide, all of other active ingredients can significantly improve the survival rate, the GSH content, and the SOD activity of the ARPE-19 cells, and the MDA activity is decreased significantly, wherein the refined Lycium barbarum leaf extract has the most prominent protective effect on the ARPE-19 cells with high-glucose induced damage.

2.2 Effects of Active Ingredients of Lycium barbarum Leaf on Gene Expression Level of ARPE-19 Cells with High Glucose-Induced Damage

In the present invention, the effects of active ingredients of Lycium barbarum leaf on the gene expression level of the ARPE-19 cells with high glucose-induced damage are investigated. Expression levels of p38, ERK and JNK genes in a MAPK pathway are analyzed by the qPCR.

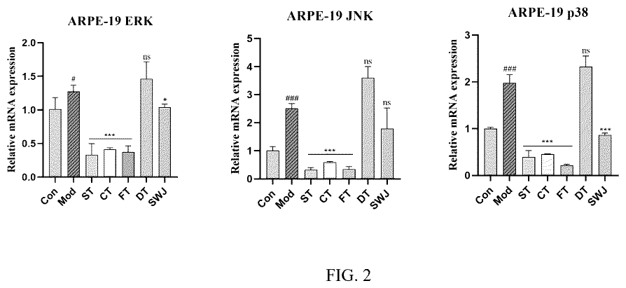

Results of the qPCR are as shown in FIG. 2, wherein the ordinates represent relative mRNA expressions. After high glucose-induced damage, the relative mRNA expressions of p38, ERK and JNK are decreased significantly compared with the blank group. After administration, except the Lycium barbarum leaf polysaccharide, other ingredients show increased relative mRNA expressions of p38, ERK and JNK, wherein the effect of the refined Lycium barbarum leaf extract is significantly better than those of other active ingredients.

2.3 Effects of Active Ingredients of Lycium barbarum Leaf on ROS Content in ARPE-19 Cells with High Glucose-Induced Damage

In the present invention, the effects of active ingredients of Lycium barbarum leaf on the ROS content in the ARPE-19 cells with high glucose-induced damage are investigated. The ROS content in the ARPE-19 cells is detected by adding a fluorescence probe (H2FCDA) in combination with a fluorescence microscope.

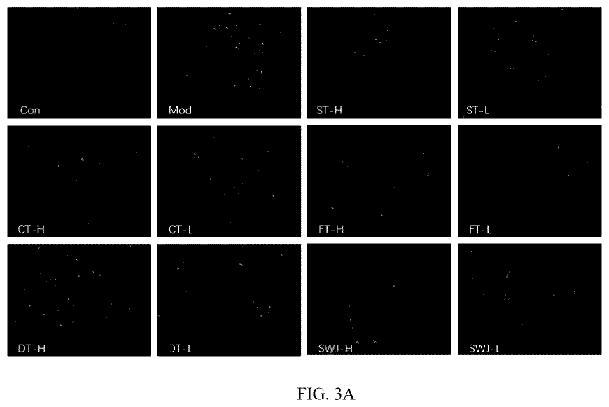

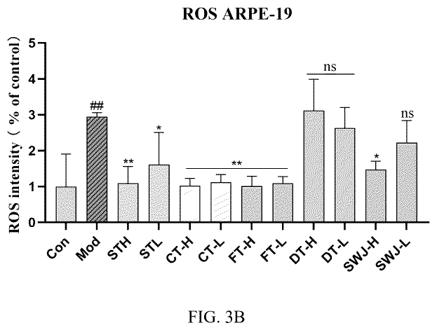

Detection results of the ROS content are as shown in FIG. 3, wherein the ordinate represents a mean fluorescence intensity. After high glucose-induced damage, the ROS fluorescence intensity is increased significantly compared with the blank group (FIG. 3A). After administration, except the Lycium barbarum leaf polysaccharide, all of other active ingredients show significantly decreased ROS fluorescence intensity, wherein the refined Lycium barbarum leaf extract achieves the best effect.

### Embodiment 3: Activity Screening Based on Zebrafish Model

### 3. Experimental Method

3dpf zebrafish embryos were cultured in a 6-well plate, with 30 zebrafish embryos in each well. A model group and an administration group were cultured in 100 mg/mL circulating water, and divided into a Con group, a Mod group, a metformin (MET) group, a ST-H group, a ST-L group, a CT-H group, a CT-L group, a FT-H group, a FT-L group, a DT-H group, a DT-L group, a SWJ-H group, and a SWJ-L group. An administration dosage for the MET group was 200 μg/ml, and a crude drug dosage was converted for other administration groups, wherein a high dose was 40 μg/mL, and a low dose was 20 μg/mL. The zebrafish embryos were cultured for three days, and the water was replaced at a fixed time every day.

3.1 Kit Detection of Effects of Active Ingredients of Lycium barbarum Leaf on Glucose Content and Oxidative Stress Level in DR Zebrafish

Wild A/B-type zebrafish embryos were cultured according to the above method, and then the zebrafish embryos were collected into Ep tubes and then homogenized, and centrifuged at 4000 rpm for 10 minutes. A supernatant was taken, and GLU and GSH levels and SOD and MDA activities in cells were detected according to a kit instruction.

3.2 qPCR Detection of Effects of Active Ingredients of Lycium barbarum Leaf on Gene Expression Level in DR Zebrafish

The cultured zebrafish embryos were collected into Ep tubes, and each tube was added with 1 mL of TRIZOL and then homogenized, and centrifuged at 4° C. and 12000× g for 10 minutes. A supernatant was taken, and other operations were as described in 2.3.

3.3 Effects of Active Ingredients of Lycium barbarum Leaf on Retinal Vessels in DR zebrafish

3dpf fli1: EGFP zebrafish embryos were cultured by the method as described in 2.4, and 0.003% phenylthiourea (PTU) was added into circulating water to inhibit melanogenesis of the zebrafish embryos. After the culture was completed, the zebrafish embryos were collected, and fixed on a glass slide. The retinal vessels in the zebrafish were photographed by a fluorescence microscope, and a retinal vascular diameter was measured by ImageJ.

### 3.4 Statistical Analysis

Data analysis and drawing were carried out by GraphPad Prism 9.4.0 software, all the results were expressed by mean±standard deviation (SD), data between groups were analyzed by one-way analysis of variance (One-way ANOVA), and the difference was statistically significant when P<0.05. (Compared with the model group, *P<0.05; ** P<0.01; *** P<0.001; and compared with the blank group, #P<0.05; ##P<0.01; ###P<0.001).

### 4. Experimental Results

4.1 Effects of Active Ingredients of Lycium barbarum Leaf on Glucose Content and Oxidative Stress Level in DR Zebrafish

In the present invention, the effects of active ingredients of Lycium barbarum leaf on the glucose level and the oxidative stress level in the DR zebrafish model are investigated. The effects of different active ingredients of Lycium barbarum leaf on the glucose content and the oxidative stress level in the DR zebrafish are detected by a GLU detection kit and a MDA, SOD and GSH detection kit in combination with a microplate reader.

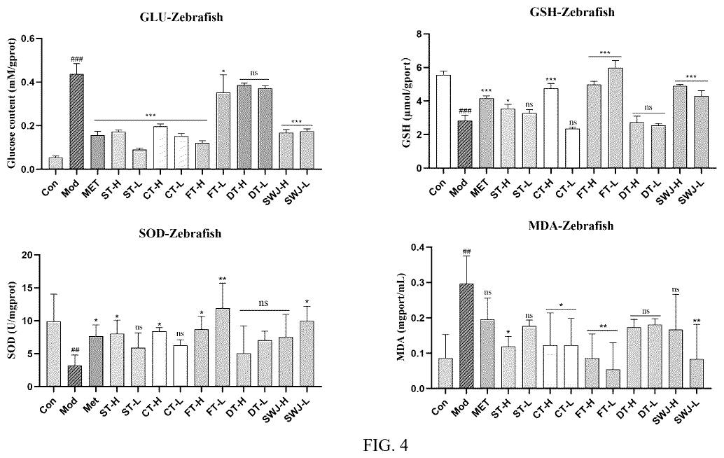

Detection results are as shown in FIG. 4, wherein the ordinates represent a glucose content, a GSH level, and MDA and SOD activities respectively. Compared with the zebrafish in the blank group, the GSH content and the SOD activity in the DR zebrafish are both decreased significantly compared with the blank group, and the glucose content and the MDA activity are increased significantly. After administration, except the Lycium barbarum leaf polysaccharide, all of other active ingredients can significantly improve the GSH content and the SOD activity in the DR zebrafish, and the glucose content and the MDA activity are decreased significantly, wherein the refined Lycium barbarum leaf extract has a lower dosage compared with the water extract and the ethanol extract according to the converted crude drug dosage, but has the most prominent effect.

4.2 Effects of Active Ingredients of Lycium barbarum Leaf on Gene Expression Level in DR Zebrafish

In the present invention, the effects of active ingredients of Lycium barbarum leaf on the gene expression level in the DR zebrafish are investigated. Expression levels of p38, ERK and JNK genes in a MAPK pathway are analyzed by the qPCR.

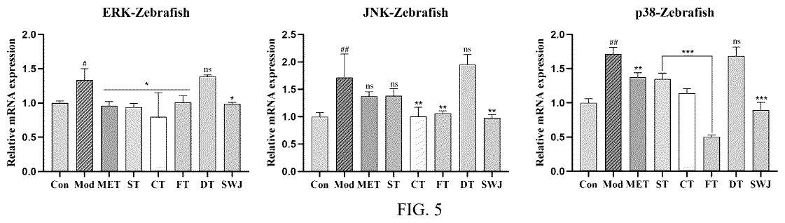

Results of the qPCR are as shown in FIG. 5, wherein the ordinates represent relative mRNA expressions. Compared with the zebrafish in the blank group, the DR zebrafish has significantly decreased relative mRNA expressions of p38, ERK and JNK compared with the blank group. After administration, except the Lycium barbarum leaf polysaccharide, other ingredients show increased relative mRNA expressions of p38, ERK and JNK. The refined Lycium barbarum leaf extract has a lower dosage compared with the water extract and the ethanol extract according to the converted crude drug dosage, but has the most prominent effect.

A comprehensive effect of refined Lycium barbarum leaf extract is significantly better than those of other active ingredients.

4.3 Effects of Active Ingredients of Lycium barbarum Leaf on Retinal Vascular Diameter in DR Zebrafish

In the present invention, the effects of active ingredients of Lycium barbarum leaf on the retinal vascular diameter in the DR zebrafish are investigated. The retinal vessels in the zebrafish are photographed by a fluorescence microscope, and a change of retinal vascular diameter in the zebrafish is analyzed in combination with ImageJ.

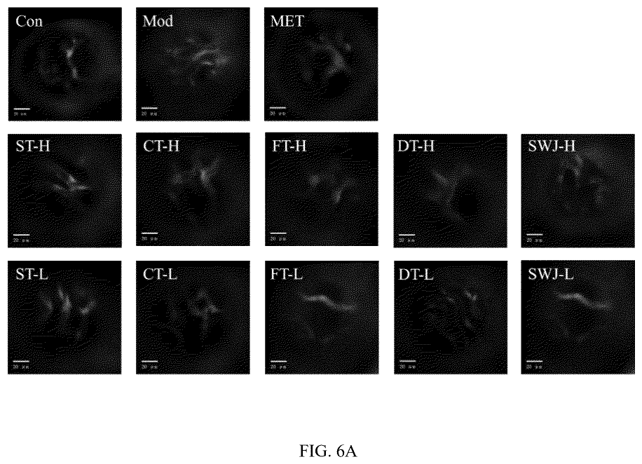

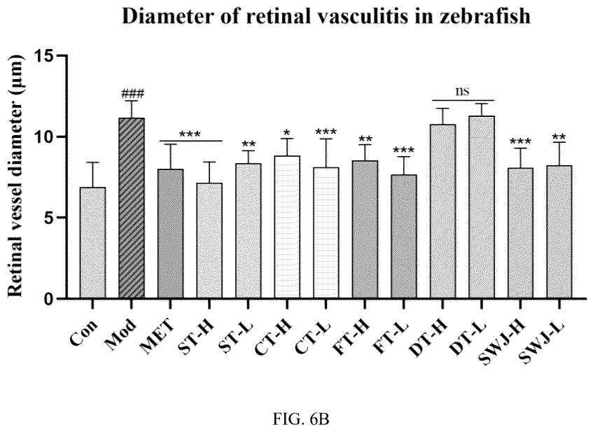

Measurement results of retinal vascular diameter are as shown in FIG. 6, wherein the ordinate represents the retinal vascular diameter in the zebrafish. Compared with the blank group, the retinal vascular diameter in the DR zebrafish is increased significantly (FIG. 6A). After administration, except the Lycium barbarum leaf polysaccharide, all of other active ingredients significantly decrease the retinal vascular diameter in the zebrafish.

According to demonstration through the in-vitro cell model and the zebrafish model, in various active ingredients of Lycium barbarum leaf, the refined Lycium barbarum leaf extract achieves the best comprehensive effect, so that the Lycium barbarum leaf water extract (ST), the Lycium barbarum leaf ethanol extract (CT), and the refined Lycium barbarum leaf extract (FT) are selected for further research.

### Embodiment 4 Activity Verification Based on Mouse Model

### 5. Experimental Method

### Construction of DR Mouse Model

Mice, 8 weeks old, were adaptively fed for 7 days, and then divided into a Con group, a Mod group, a metformin (MET) group, a ST-H group, a ST-L group, a CT-H group, a CT-L group, a FT-H group, and a FT-L group. The blank group was fed with common feed, while the model group and the administration group were fed with high-glucose and high-fat feed, and a body weight of the mice was measured every week. After 8 weeks of continuous feeding, the mice in the model group and the mice in the administration group were intraperitoneally injected with 1% STZ solution (STZ dissolved in 0.1 mol/L sodium citrate solution) (60 mg/kg) for 3 consecutive days, a blood glucose level in the mice was detected 72 hours after injection, and when fasting blood-glucose was ≥16.7 mM, the modeling was successful. The mice in blank group were injected with the same volume of sodium citrate buffer. After the modeling was completed, the mice were fed with high-glucose and high-fat feed for 8 consecutive weeks at the same time, and intragastrically administered with a corresponding drug every day, and a blood glucose level in the mice was detected every week. An administration dosage for the MET group was 400 mg/kg/d, and a crude drug dosage was converted for other administration groups, wherein a high dose was 4.5 g/kg/d and a low dose was 2.25 g/kg/d.

5.1 Effects of Different Active Ingredients of Lycium barbarum Leaf on Retinal Thickness and Retinal Vascular Density of DR Mice

24 hours after the last administration, the mice were anesthetized by intraperitoneal injection of 1% pentobarbital sodium (at dosages of 50 mg/kg and 0.5 mL/100 g). The retinal thickness and the retinal vascular density of the mice were detected by Spectralis OCT and OCTA, and results of the retinal thickness and the retinal vascular density were analyzed by ImageJ.

### 5.2 Statistical Analysis

Data analysis and drawing were carried out by GraphPad Prism 9.4.0 software, all the results were expressed by mean±standard deviation (SD), data between groups were analyzed by one-way analysis of variance (One-way ANOVA), and the difference was statistically significant when P<0.05. (Compared with the model group, *P<0.05; ** P<0.01; *** P<0.001; and compared with the blank group, #P<0.05; ##P<0.01; ###P<0.001).

### 6. Experimental Results

6.1 Effects of Active Ingredients of Lycium barbarum Leaf on Body Weight and Blood Glucose Level of DR Mice

In the present invention, the effects of active ingredients of Lycium barbarum leaf on the body weight and the blood glucose level of the DR mice are investigated. A body weight change of the mice is monitored every week during feeding, and a blood glucose level change of the mice is monitored by a blood glucose meter after STZ injection.

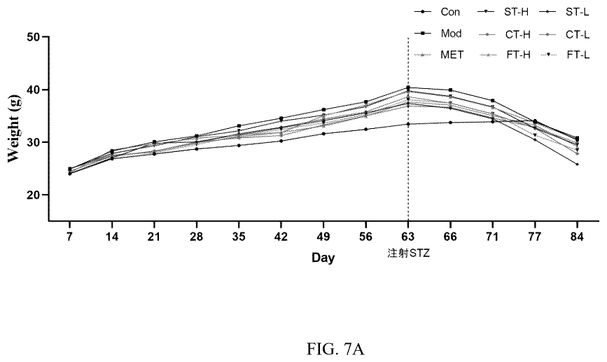

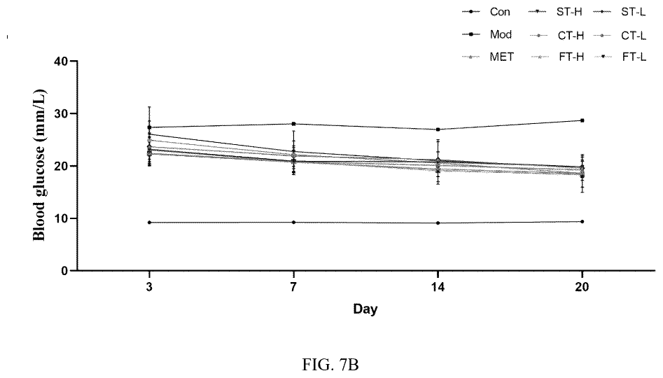

Results of body weight change and blood glucose level change of the DR mice are as shown in FIG. 7. In FIG. 7(A), the abscissa represents a feeding date and the ordinate represents a body weight of the mice; and in FIG. 7(B), the abscissa represents a feeding date and the ordinate represents a blood glucose level of the mice. Compared with the blank group, the body weight of the mice in the model group is higher. After STZ injection, the body weights of the mice in the model group and the mice in the administration group begin to be decreased, while the body weight of the mice in the blank group is still increased steadily. After injection of STZ, the blood glucose levels of the mice in the model group and the mice in the administration group are much higher than that of the mice in the blank group. After administration, the blood glucose level of the DR mice is decreased steadily.

6.2 Effects of Active Ingredients of Lycium barbarum Leaf on Retinal Thickness and Retinal Vascular Density of DR Mice

In the present invention, the effects of active ingredients of Lycium barbarum leaf on the retinal thickness and the retinal vascular density of the DR mice are investigated. The retinal thickness and the retinal vascular density of the mice are detected by Spectralis OCT and OCTA, and results of retinal thickness change and retinal vascular density change of the mice are analyzed by ImageJ.

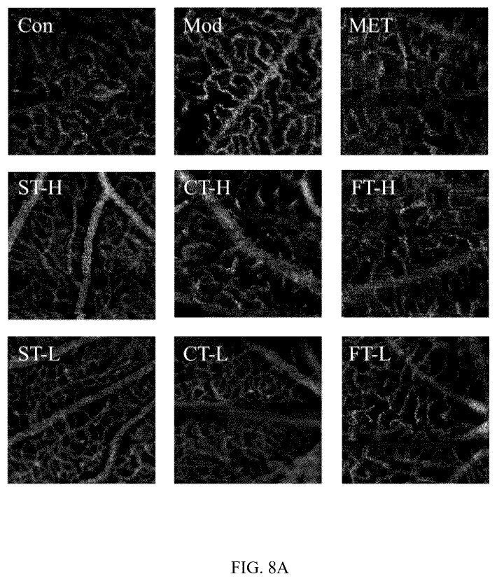

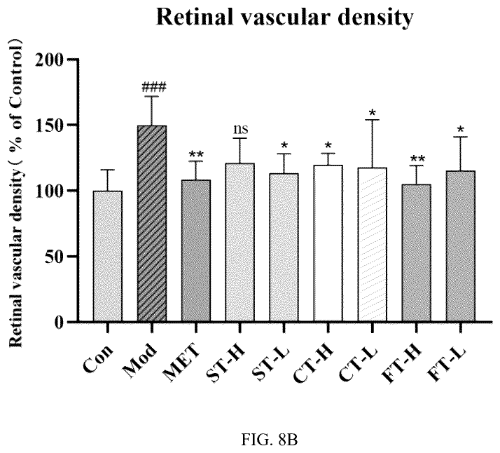

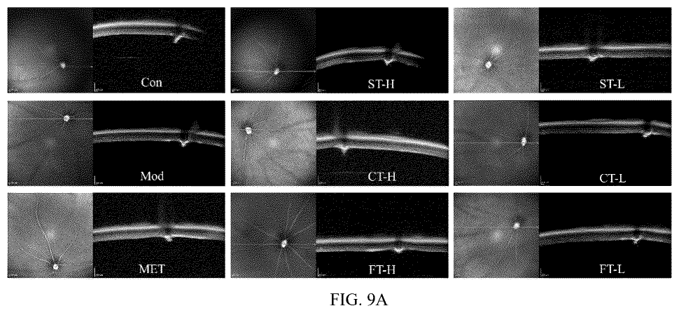

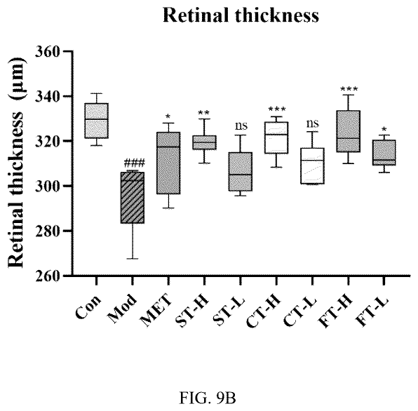

Measurement results of retinal thickness and retinal vascular density are as shown in FIG. 8 and FIG. 9, wherein the ordinates represent a retinal thickness and a retinal vascular density of mice respectively. Compared with the blank group, the retinal thickness of the DR mice is decreased significantly (FIG. 8) and the retinal vascular density is increased significantly. After administration, all the active ingredients increase the retinal thickness of the mice and decrease the retinal vascular density.

By comparing the improvement effects of different active ingredients of Lycium barbarum leaf on diabetic retinopathy, it is found that the refined Lycium barbarum leaf extract is the best active ingredient, so that the refined Lycium barbarum leaf extract may be separated and applied to the field of DR treatment alone, so as to realize the efficient and comprehensive utilization of Lycium barbarum leaf resources.

The above embodiments are only used to explain the technical concepts and features of the present invention, and are intended to enable those skilled in the art to understand and implement the contents of the present invention, without limiting the scope of protection of the present invention. All equivalent changes or modifications made according to the spirit of the present invention should be included in the scope of protection of the present invention.

What is claimed is:

1. A method for treating a disease comprising a step of administering a Lycium barbarum leaf extract to a subject in need, wherein the disease is selected from the group consisting hyperglycemia, diabetes and diabetic retinopathy.

2. The method according to claim 1, wherein the Lycium barbarum leaf extract is selected from the group consisting of a Lycium barbarum leaf water extract, a Lycium barbarum leaf ethanol extract, a Lycium barbarum polysaccharide, a Lycium barbarum alkaloid and a refined Lycium barbarum leaf extract.

3. The method according to claim 2, wherein the refined Lycium barbarum leaf extract is prepared by the following method: dried Lycium barbarum leaves are weighed, added with ethanol, and heated and refluxed for extraction to obtain a Lycium barbarum leaf ethanol extracting solution, the extracting solution is concentrated under a reduced pressure to a suitable volume, then fully adsorbed with D101 macroporous resin, and eluted with distilled water, 10% ethanol and 30% ethanol respectively, an eluate is discarded, the rest is eluted with 70% ethanol which serves as an eluent, and an eluate is collected, concentrated under a reduced pressure, and freeze-dried to obtain the refined Lycium barbarum leaf extract.

4. The method according to claim 2, wherein the Lycium barbarum leaf water extract is prepared by the following method: dried Lycium barbarum leaves are weighed, added with distilled water, and heated and refluxed for extraction, and extracting solutions are combined, concentrated under a reduced pressure, and freeze-dried to obtain the Lycium barbarum leaf water extract.

5. The method according to claim 2, wherein the Lycium barbarum leaf ethanol extract is prepared by the following method:
dried Lycium barbarum leaves are weighed, added with ethanol, and heated and refluxed for extraction, and Lycium barbarum leaf ethanol extracting solutions are combined, concentrated under a reduced pressure, and freeze-dried to obtain the Lycium barbarum leaf ethanol extract; and
a herb residue obtained after ethanol extraction is air-dried, added with distilled water, and heated and refluxed for extraction to obtain an extracting solution, the extracting solution is concentrated under a reduced pressure, then added with a certain amount of anhydrous ethanol, and allowed to stand overnight, a supernatant is discarded, the rest is dried to obtain a crude polysaccharide, and the crude polysaccharide is deproteinized by a Sevage method to obtain the Lycium barbarum polysaccharide.

6. The method according to claim 2, wherein the Lycium barbarum alkaloid is betaine.

7. The method according to claim 1, wherein the Lycium barbarum leaf extract and a pharmaceutically acceptable carrier are made into a drug in a form of a tablet, a capsule, a granule, a pill, a mixture, an oral liquid, or an ointment.

8. The method according to claim 1, wherein the diabetic retinopathy is caused by type I diabetes or type II diabetes.
